SEQUENCE LISTING (18 sequences)

SEQ ID NO: 1 - DNA, 20 bp, Mus musculus
  source 1..20: mol_type = other DNA
ctatgggatcagccagaaag

SEQ ID NO: 2 - DNA, 19 bp, Mus musculus
  source 1..19: mol_type = other DNA
agtcaaaggcatccaccaa

SEQ ID NO: 3 - DNA, 25 bp, Mus musculus
  source 1..25: mol_type = other DNA
cacagctgccttctgaaatgtgtgg

SEQ ID NO: 4 - DNA, 25 bp, Mus musculus
  source 1..25: mol_type = other DNA
tttctacgtggcccggttctaattc

SEQ ID NO: 5 - DNA, 19 bp, Mus musculus
  source 1..19: mol_type = other DNA
tctaggcaatgccgttcac

SEQ ID NO: 6 - DNA, 19 bp, Mus musculus
  source 1..19: mol_type = other DNA
gagcacatgggcaccatac

SEQ ID NO: 7 - DNA, 21 bp, Mus musculus
  source 1..21: mol_type = other DNA
agccatggaggaggtggtgat

SEQ ID NO: 8 - DNA, 21 bp, Mus musculus
  source 1..21: mol_type = other DNA
gtgtgcctgcttggggtggac

SEQ ID NO: 9 - DNA, 21 bp, Mus musculus
  source 1..21: mol_type = other DNA
aaagccgctcgcaagagtgcg

SEQ ID NO: 10 - DNA, 20 bp, Mus musculus
  source 1..20: mol_type = other DNA
acttgcctcctgcaaagcac

SEQ ID NO: 11 - DNA, 19 bp, Mus musculus
  source 1..19: mol_type = genomic DNA
acaaggcctcagggtacca

SEQ ID NO: 12 - DNA, 20 bp, Mus musculus
  source 1..20: mol_type = other DNA
gccgaaagaagcccttacag

SEQ ID NO: 13 - DNA, 21 bp, Mus musculus
  source 1..21: mol_type = other DNA
gctgttatgggtgaaactctg

SEQ ID NO: 14 - DNA, 23 bp, Mus musculus
  source 1..23: mol_type = other DNA
gaataataaggtggagatgcagg

SEQ ID NO: 15 - DNA, 20 bp, Mus musculus
  source 1..20: mol_type = other DNA
agtcatcatcacccaagagc

SEQ ID NO: 16 - DNA, 19 bp, Mus musculus
  source 1..19: mol_type = other DNA
gggatgacatacatggtgc

SEQ ID NO: 17 - DNA, 20 bp, Mus musculus
  source 1..20: mol_type = other DNA
cacttcatcaaggcagactc

SEQ ID NO: 18 - DNA, 20 bp, Mus musculus
  source 1..20: mol_type = other DNA
cggtagcgcttctcaatggc

(19) United States
(12) Patent Application Publication
(10) Pub. No.: US 2025/0295709 A1
(43) Pub. Date: Sep. 25, 2025

(54) POSTBIOTIC PREPARATION FOR PREVENTING OR TREATING OBESITY

(71) Applicant: GENBIOMA APLICACIONES, S.L., NOÁIN (ES)

(72) Inventors: JOSUNE AYO MARTÍNEZ, URDULIZ (ES); MARÍA ONECA AGURRUZA, PAMPLONA (ES); IGNACIO ITURRIA GALLEGO, SAN SEBASTIÁN (ES); DEYAN YAVOROV DAYLIEV, BURLADA (ES); MIGUEL ÁNGEL BARAJAS VÉLEZ, PAMPLONA (ES); IGNACIO JOSÉ ENCÍO MARTÍNEZ, GORRAIZ (ES); MIRIAM ARAÑA CIORDIA, NUEVO ARTICA (ES); MIRIAM CABELLO OLMO, SEVILLA (ES)

(21) Appl. No.: 18/978,675

(22) Filed: Dec. 12, 2024

(30) Foreign Application Priority Data
Mar. 21, 2024 (EP) 24382308.5

(51) Int. Cl. A61K 35/744; A61K 9/00; A61K 33/30; A61K 45/06; A61P 3/04

(52) U.S. Cl. CPC A61K 35/744; A61K 9/0056; A61K 33/30; A61K 45/06; A61P 3/04

(57) ABSTRACT

A postbiotic preparation of Pediococcus acidilactici (P. acidilactici), for use in preventing and/or treating obesity, in reducing body fat, in preventing fat accumulation, in reducing appetite, in reducing food intake, or in preventing weight gain.

### CROSS-REFERENCE TO RELATED APPLICATIONS

This application claims the benefit of European Patent Application EP24382308.5, filed on Mar. 21, 2024, which is incorporated herein by reference in its entirety.

The material in the xml file, named “P6548US00seqlist.xml”, created Dec. 12, 2024, file size of 3,170,134 bytes, is hereby incorporated by reference.

### TECHNICAL FIELD

The present invention relates to compositions for promoting health, in particular compositions comprising a postbiotic preparation of Pediococcus acidilactici for preventing or treating obesity.

### BACKGROUND ART

In recent years, there has been a gradual rise in global obesity rates. The World Health Organization (WHO) has characterized the rapid proliferation of obesity as an “infectious disease,” coining the term “Globesity.”

Obesity escalates the risk of various health issues, leading to complications such as hypertension, hyperlipidemia, cardiovascular disease, sleep apnea, and even cancer. Consequently, obesity diminishes quality of life and may result in premature death, with the life expectancy of morbidly obese individuals significantly lower than that of those with normal weight. While genetic factors may play a role in obesity development, the epidemic is primarily attributed to high-calorie diets and sedentary lifestyles. Lifestyle modifications are, therefore, crucial in preventing obesity and its associated complications.

Although low-calorie diets and regular exercise are traditional methods for weight reduction and obesity treatment, their implementation is challenging, and their effectiveness is limited due to the body's adaptive physiological mechanisms that preserve energy storage. While some drugs like glucagon-like peptide 1 (GLP-1) analogues such as semaglutide and liraglutide, GLP-1/GIP dual agonists such as tirzepatide, gastrointestinal lipase inhibitors such as orlistat, or appetite suppressants such as phentermine or topiramate, have been approved for long-term obesity treatment, they may have side effects, including gastrointestinal disorders. Besides, these are expensive medicaments requiring prescription. Bariatric surgery, which significantly reduces body weight, is available for some individuals, but it is neither accessible nor suitable for many others.

Hence, there is a need to develop new strategies for reducing body weight and fat accumulation.

### SUMMARY OF INVENTION

The inventors have found that consumption of a postbiotic preparation of bacteria of the species Pediococcus acidilactici (P. acidilactici) has the surprising effect of preventing weight gain and improving obesity-related parameters with the consequence that these bacteria are useful for preventing and/or treating obesity or, in general, for preventing fat accumulation and/or reducing body fat.

Thus, in first aspect the invention refers to a postbiotic preparation of Pediococcus acidilactici for use in preventing and/or treating obesity, in reducing body fat, in preventing fat accumulation, in reducing appetite, in reducing food intake, or in preventing weight gain. This aspect can be alternatively formulated as the use of a postbiotic preparation of Pediococcus acidilactici for the manufacture of a medicament for preventing and/or treating obesity, in reducing body fat, in preventing fat accumulation, in reducing appetite, in reducing food intake, or in preventing weight gain. The invention also refers to a method for preventing and/or treating obesity, reducing body fat, preventing fat accumulation, reducing appetite, reducing food intake, or preventing weight gain in an animal in need thereof, including a human, comprising administering to said animal in need thereof an effective amount of a postbiotic preparation of Pediococcus acidilactici.

As shown in the examples below, a preparation of heat-inactivated Pediococcus acidilactici surprisingly was able to prevent weight gain and improved several obesity-related parameters, in particular, the preparation reduced adipocyte size, reduced food efficiency, reduced steatosis, and reduced leptin in mice fed a high-fat diet when compared to controls fed the high-caloric diet but not receiving the inactivated Pediococcus acidilactici. Even more surprisingly, the inactivated Pediococcus acidilactici preparation (postbiotic preparation) achieved better results in preventing weight gain and improving obesity-related parameters when compared to live P. acidilactici (probiotic preparation). A probiotic preparation comprising Pediococcus acidilactici was disclosed in WO2021123355 for glycemic control. However, this document does not disclose any effect in controlling obesity. Moreover, the obesity-controlling activity of the postbiotic preparation of the present invention is surprising, since it is usually considered that inanimate bacteria have milder effects than live, metabolically active bacteria. Indeed, WO2021123355 discloses that heat-killed P. acidilactici CECT 9879 unexpectedly showed equivalent glycaemia-controlling effects when compared to live cells. The fact that the postbiotic preparation of the present invention shows better obesity-controlling effects when compared to its life counterpart is even more surprising.

A second aspect of the invention refers to a composition comprising a postbiotic preparation of P. acidilactici for use in preventing and/or treating obesity, in reducing body fat, in preventing fat accumulation, in reducing appetite, in reducing food intake, or in preventing weight gain. Again, this aspect may be reformulated as the use of a composition comprising a postbiotic preparation of P. acidilactici for the manufacture of a medicament for preventing and/or treating obesity, in reducing body fat, in preventing fat accumulation, in reducing appetite, in reducing food intake, or in preventing weight gain. The invention also refers to a method for preventing and/or treating obesity, in reducing body fat, in preventing fat accumulation, in reducing appetite, in reducing food intake, or in preventing weight gain in an animal in need thereof, including a human, comprising administering to said animal in need thereof an effective amount of a composition comprising a postbiotic preparation of P. acidilactici.

The postbiotic preparation of the first aspect and the composition of the second aspect possess several advantages when compared with live or metabolically active bacterial preparations while maintaining or even improving their anti-obesity effect. In particular, the postbiotic preparation has better stability, both during industrial processes and storage. This means that, for example, the postbiotic preparation or composition may be subjected to very harsh conditions imposed by conventional industrial processes, such as spray-drying or extrusion, without negatively affecting their activity. The postbiotic preparation also has the advantage of not requiring oxygenation. The postbiotic preparation is also safe. In this sense, it is noted that P. acidilactici is generally recognized as safe (GRAS) and thus approved for animal and human consumption. Last but not less, the postbiotic preparation contain inanimate microorganisms, bypassing the problem of acquiring antibiotic resistance genes and virulence factors, which is growing global health threat that requires urgent attention.

The postbiotic preparation of the first aspect, or the composition of the second aspect also have some advantages when compared to drugs for the prevention or treatment of obesity, particularly related to the lack of side effects and the reduced cost. However, it may be convenient to use the postbiotic preparation or composition of the invention in combination with lifestyle changes (e.g., diet modifications) and/or other drugs that have anti-obesity properties in order to obtain a synergistic effect.

### BRIEF DESCRIPTION OF DRAWINGS

FIG. 1. Nile Red Staining of pA1c®HI-supplemented worms at different doses in a glucose-loaded (10 mM) medium. (A) % of fat accumulation with respect of the control. (B) % of fat reduction with respect to the control.

FIG. 2. Nile Red Staining of pA1c®HI- and pA1c®-supplemented worms in a glucose-loaded (10 mM) medium. (A) % of fat accumulation with respect to the control. (B) % of fat reduction with respect to the control.

FIG. 3. Microscope observation of the presence of eggs (white arrows) and L1 larvae (grey arrows) in control−, control+glucose−, pA1c®+glucose- and pA1c®HI+glucose-supplemented plates.

FIG. 4. Classification of worms with respect of daf-16: GFP localization under different conditions: Control, Control+Glucose, pA1c®+glucose and pA1c®HI+glucose.

FIG. 5. Nile Red Staining of pA2cHI-supplemented worms in a glucose loading medium. (A) % of fat accumulation compared to the control. (B) % of fat reduction compared to the control.

FIG. 6. Effect of pA1c® and pA1c®HI on (A) body weight gain (%), (B) total weight gain (%), (C) food intake, and (D) food efficiency. 9-10 animals/group. * p<0.05 and *p<0.01.

FIG. 7. Effect of pA1c® and pA1c®HI on (A) adipocyte area and (B) adipocyte diameter. (C) Representative images of H&E-stained adipose tissue sections (scale bars=100 μm). 9-10 animals/group. ** p<0.01.

FIG. 8. Effect of pA1c® and pA1c®HI on (A) glycemia, (B) serum leptin and (C) colonic GLP-1. n=8-10 animals/group. ^{$$}p<0.01 vs Co; ^{$$$}p<0.001 vs Co; *p<0.05; **p<0.01 and ***p<0.001. ns: no significant differences.

FIG. 9. Impact of pA1c® and pA1c®HI on (A) hepatic steatosis, and (B) ALT serum levels. n=9-10 animals/group. *p<0.05, **p<0.01 and ***p<0.001. ALT: alanine aminotransferase; AST alanine: aspartate aminotransferase; ns: no significant differences.

FIG. 10. Impact of pA1c® and pA1c®HI on (A) hepatic and (B) adipose tissue gene expression. n=8 animals/group. * p<0.05, ** p<0.01 and ** p<0.001. Acox: Peroxisomal acyl-coenzyme A oxidase 1; Cpt1a: Carnitine palmitoyl transferase deficiency-type 1; CD36: Cluster of differentiation 36; Fasn: fatty acid synthase; ns: no significant differences; Pparα: peroxisome proliferator-activated receptor α; Pparγ: peroxisome proliferator-activated receptor γ; Srebp: sterol regulatory element-binding protein.

### DETAILED DESCRIPTION OF THE INVENTION

### Definitions

According to the International Scientific Association of Probiotics and Prebiotics (ISAPP), the term “postbiotic” refers to a preparation of inanimate microorganisms and/or their components that confers a health benefit on the host (beyond basic nutrition). “Inanimate microorganisms” is understood as lifeless microorganisms. The word ‘components’ includes microbial cell components, cell wall components or other structures, as well as microbial metabolites or end products of growth on the specified matrix produced during growth and/or fermentation. “Bacterial metabolites” includes all molecules produced or modified by the bacteria as a result of bacterial metabolism during growth.

Examples of bacterial metabolites include all organic acids, inorganic acids, bases, proteins and peptides, enzymes and co-enzymes, amino acids and nucleic acids, carbohydrates, lipids, glycoproteins, lipoproteins, glycolipids, vitamins, all bioactive compounds, metabolites containing an inorganic component, and all small molecules, for example, nitrous molecules or molecules containing a sulfurous acid. Thus, a postbiotic preparation may contain whole inanimate cells, lysed cells, or even a cell-free extract obtained from the microbial cells.

The term “probiotic” in the sense of the present invention is defined as a life microorganism that, when consumed in adequate amounts, exerts health benefits to the host beyond inherent basic nutrition.

The term “prebiotics” is generally defined as non-digestible food ingredients that beneficially affect the host by selectively stimulating the growth and/or activity of one or a limited number of bacteria in the colon, and thus improve host health. Typically, prebiotics are carbohydrates (such as oligosaccharides), but the definition does not preclude non-carbohydrates.

“Medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need marketing approval, but may include nutraceuticals and natural remedies.

The term “effective amount” as used herein, means an amount of an active agent high enough to deliver the desired benefit, but low enough to avoid serious side effects within the scope of medical judgment. This term refers to that amount of the active component of the pharmaceutical composition which is sufficient to cause the prevention and/or treatment, of a disease or pathological condition of interest in the mammal, particularly 25 in humans. The effective amount could vary according to the activity of the strain of the invention and its active agents, according to its metabolic stability, in any presentation form, and among other factors, but it can be determined by a person skilled in the art according to his/her own knowledge and this description.

The term “mutants” refer to strains obtained using the bacterial strains of the invention as starting material. A “mutant” of the strain is also understood according to the invention as a “variant” or “derivative”.

In this specification the term “preventing” refers to preventing the specified condition from occurring in an animal which may be predisposed to the condition and/or may be exposed to conditions that favor developing of the condition but does not yet experience or display the pathology or symptomatology.

The term “treatment” refers to inhibiting the condition in a mammal that is experiencing or displaying the pathology or symptomatology of the condition (i.e., arresting further development of the pathology and/or symptomatology) or ameliorating the condition in a mammal that is experiencing or displaying the pathology or symptomatology of the condition (i.e., reversing the pathology and/or symptomatology or stopping or slowing its progression). The term “treatment” encompasses prophylactic treatment.

“Obesity” is a condition in which the natural energy reserve, stored in the fatty tissue of animals, in particular humans and other mammals, is increased to a point where it is associated with certain health conditions or increased mortality. The expression “excessive fat accumulation” is understood as having more body fat than is optimally healthy. “Excessive fat accumulation” may be also termed “overweight”. Obesity and excessive fat accumulation are usually defined by the Body mass index of an individual. “Body mass index” or “BMI” means the ratio of weight in kg divided by the height in meters, squared. “Obesity” is generally defined for a human adult as having a BMI greater than 30. “Overweight” is generally considered for human adults as BMI from 25 to 29.9. For human children above 2 years of age, the BMI is plotted on a BMI vs. age growth chart (for either girls or boys) to obtain a percentile ranking. For children below 2 years (infants) what is plotted is the weight-for-length instead of the BMI. Percentiles and z-scores are the most commonly used indicator to assess the size and growth patterns of individual children. The percentile indicates the relative position of the child's BMI or weight-for-length in a population of individuals with the same sex and age. The z-score indicates the number of standard deviations (SDS) that one individual deviates from the mean of a population with the same sex and age. Human children are considered overweight if their BMI or weight-for-length percentile is located between the 85th and 95th percentile, or the z-score between 1 and 2 SDS. Human children are considered obese if their BMI or weight-for-length percentile is located on or above the 95th percentile, or z-score equal or higher than 2 SDS.

As used herein, the term “functional food” means food which is capable of providing not only a nutritional effect, but is also capable of delivering a further beneficial effect to consumer. Accordingly, functional foods are ordinary foods that have components or ingredients (such as those described herein) incorporated into them that impart to the food a specific functional—e.g. medical or physiological benefit-other than a purely nutritional effect. In the sense of the present invention the term food or functional food includes solid foods as well as beverages.

The term “nutraceutical” means a composition which is capable of providing not only a nutritional effect and/or a taste satisfaction, but is also capable of delivering a therapeutic (or other beneficial) effect to the consumer. Nutraceuticals cross the traditional dividing lines between foods and medicine.

The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

In the sense of the present disclosure the expression “consisting essentially of” means that specific further components can be present, namely those not materially affecting the essential characteristics of the composition.

### Postbiotic Preparation

As mentioned above, the first aspect of the invention refers to a postbiotic preparation of P. acidilactici for use in preventing and/or treating obesity, in reducing body fat, in preventing fat accumulation, in reducing food intake, or in preventing weight gain. Attending to the currently accepted definition of “postbiotic”, this aspect can be alternatively worded as a preparation comprising or consisting essentially of inanimate P. acidilactici cells and/or their components for use in preventing and/or treating obesity, in reducing body fat, in preventing fat accumulation, in reducing appetite, in reducing food intake, or in preventing weight gain.

In one embodiment, the postbiotic preparation comprises inanimate P. acidilactici cells. Methods to kill bacteria are known in the art. The most common processes used for microbial inactivation are thermal (pasteurization, sterilization, and ohmic heating) and non-thermal (pulsed electric fields, ultrasound, irradiation, and supercritical carbon dioxide) processes. Some methods maintain the structural integrity of the bacterial cells. Non limiting examples of these methods are phenolization, irradiation and formalin or formaldehyde treatment (Fan et at, J Food Prot 2017; Levinson et al., JAMA 1944; Hankaniemi et al, Antiviral Research 2019). One example of killing the bacterial cells without destroying their structural integrity is treating the bacteria with phenol 0.5% (w/v) during 12 h at room temperature. In a particular embodiment, the P. acidilactici cells have been killed by heat treatment, such as submitting the cells to a temperature from 60 to 120° C. during 1 to 60 minutes. Stirring may be applied during the inactivation (killing) procedure.

In the sense of the present invention, the postbiotic preparation is obtainable by submitting P. acidilactici cells to a treatment to kill said cells, such as any of the methods disclosed above (heat, phenolization, etc). In one embodiment, the invention refers to a postbiotic preparation of P. acidilactici for use in preventing and/or treating obesity, in reducing body fat, in preventing fat accumulation, in reducing food intake, or in preventing weight gain, wherein the postbiotic preparation is obtainable by submitting P. acidilactici cells to a treatment to kill said cells, such as any of the methods disclosed above (heat, phenolization, etc).

In one embodiment, the postbiotic preparation comprises P. acidilactici cells, wherein at least 70% of the cells are inanimate. In another embodiment, at least 80% of the P. acidilactici cells in the postbiotic preparation are inanimate. In a particular embodiment, at least 90% of the P. acidilactici cells in the postbiotic preparation are inanimate. In another particular embodiment, at least 95% of the P. acidilactici cells in the postbiotic preparation are inanimate. In another particular embodiment, at least 96%, 97%, 98%, or 99% of the P. acidilactici cells in the postbiotic preparation are inanimate. In another particular embodiment, 100% of the P. acidilactici cells in the postbiotic preparation are inanimate.

In another embodiment, the postbiotic preparation comprises a cell lysate of P. acidilactici. A cell lysate may be obtained by subjecting the bacterial cells to disruption, for example, by means of a French press. In another embodiment, the postbiotic preparation of the first aspect comprises a cell-free extract of P. acidilactici. A cell-free extract is obtainable by disrupting the bacterial cells and removing the cell wall and membrane components, for example by centrifugation.

In another embodiment the postbiotic preparation comprises a culture's supernatant of P. acidilactici. Said culture's supernatant may be obtainable by culturing P. acidilactici in a suitable medium and removing the bacterial cells, for example, by centrifugation. The culture's supernatant contains metabolites secreted by the bacterial cells during their growth, which may have obesity-controlling effects. Said metabolites may be identified, for example, by high performance liquid chromatography (HPLC), matrix-assisted laser desorption/ionization-time-of-flight (MALDI-TOF)-MS, or proton nuclear magnetic resonance (NMR) spectroscopy.

Optionally, the postbiotic preparation may be subjected to a dehydration process. The dehydration can be carried out by a lyophilization process, but the slurry can also be dried by fluidized bed drying, or by atomization, spray-drying, etc. Although the postbiotic preparation is particularly resistant to harsh conditions, including high temperature and/or pressure, cryoprotectants and/or lyophilization additives may be added. The postbiotic preparation may be also be directly frozen. Preservatives such as glycerol may be used when freezing.

### Bacterial Strains

The postbiotic preparation described in the present invention is obtained from bacteria that belong to the species P. acidilactici. This species is generally recognized as safe (GRAS) and thus approved for animal and human consumption.

In one embodiment, the postbiotic preparation of the first aspect comprises a strain of Pediococcus acidilactici deposited at the “Colección Española de Cultivos Tipo” (CECT) with identification reference CECT 9879. The strain of P. acidilactici CECT 9879 of the invention, was also isolated from cow stool in Navarra region (Spain) and was deposited, according to the Budapest Treaty, on 05.09.2019 in the “Colección Española de Cultivos Tipo” (CECT), by the depositor Pentabiol S. L., sited at Polígono Industrial Noain-Esquiroz, Calle S, Nave 4, 31191 Esquíroz, Navarra (Spain). The strain received the accession number CECT 9879 after the International Authority of Deposit declared the strain as viable.

In another embodiment, the postbiotic preparation of the first aspect comprises a strain of Pediococcus acidilactici deposited at the “Colección Española de Cultivos Tipo” (CECT) with identification reference CECT 9889. The strain of P. acidilactici CECT 9889 of the invention, was isolated from cow stool in Navarra region (Spain) and was deposited, according to the Budapest Treaty, on May 9,2019 in the “Colección Española de Cultivos Tipo” (CECT), by the depositor Pentabiol S. L., sited at Polígono Industrial Noain-Esquiroz, Calle S, Nave 4, 31191 Esquíroz, Navarra (Spain). The strain received the accession number CECT 9889 after the International Authority of Deposit declared the strain as viable.

In another embodiment, the postbiotic preparation of the first aspect comprises a strain of Pediococcus acidilactici deposited at the “Colección Española de Cultivos Tipo” (CECT) with identification reference CECT 9967, and combinations thereof. The strain of P. acidilactici CECT 9967 of the invention, was isolated from cow stool in Navarra region (Spain) and was deposited, according to the Budapest Treaty, on Jan. 10, 2019 in the “Colección Española de Cultivos Tipo” (CECT), by the depositor Pentabiol S. L., sited at Polígono Industrial Noain-Esquiroz, Calle S, Nave 4, 31191 Esquíroz, Navarra (Spain). The strain received the accession number CECT 9967 after the International Authority of Deposit declared the strain as viable.

The depositor Pentabiol S. L. authorised GENBIOMA APLICACIONES, S. L. sited in Polígono Industrial Noain-Esquiroz Calle S, Nave 4, 31191 Esquíroz, Navarra (Spain) to refer to the deposited biological material in the European patent application having the representative's reference number P6548EP00 and gave their unreserved and irrevocable consent to the deposited material being made available to the public as from the date of filing of this patent application.

In some embodiments, the postbiotic preparation comprises a combination of inanimate cells of P.acidilactici and/or their components. In one embodiment, the postbiotic preparation comprises inanimate cells of CECT 9879 and/or its components and inanimate cells of P.acidilactici CECT 9889 and/or its components. In other embodiment, the postbiotic preparation comprises inanimate cells of CECT 9879 and/or its components and inanimate cells of P.acidilactici CECT 9967 and/or its components. In other embodiment, the postbiotic preparation comprises inanimate cells of CECT 9889 and/or its components and inanimate cells of P.acidilactici CECT 9967 and/or its components. In other embodiments the postbiotic preparation further comprises inanimate cells of another P.acidilactici strain and/or its components. In other embodiments the postbiotic preparation further comprises inanimate cells of another bacterial strain and/or its components.

An expert in the art will understand that using the bacterial strains of the invention as starting material it is routinely possible to obtain, for example by spontaneous mutation or directed mutagenesis, mutants that retain the characteristics and relevant advantages of the strains of the invention. It would be apparent to a skilled person that such mutant strains may be used in the present invention as obvious alternatives (equivalents) to the strains disclosed herein for obtaining the postbiotic preparation. Methods for obtaining mutants of a given bacterial strain are well known in the art. Examples can be found in (Sambrook, J. and Russell, D W “Molecular Cloning: A Laboratory Manual”, Chapter 13, “Mutagenesis”, Cold Spring Harbor, 3rd Ed., 2001). Mutants may be obtained by genetic engineering methods, such as site directed mutagenesis. Mutants may also be obtained by subjecting the strains of the invention to mutagenic conditions, such as UV light, or to stress conditions, which may result in obtaining what is usually called “spontaneous mutants”.

### Culture

As mentioned above, the postbiotic preparation is obtainable by submitting the bacterial cells to a process for inactivating the same. In any case, the first step to obtain a postbiotic preparation is to obtain the bacterial cells of interest (in the present invention, P. acidilactici cells).

The bacterial cells may be obtained by methods well known to a skilled person. For example, a process for preparing a bacterial cell suspension of P. acidilactici comprises: (i) inoculating the P. acidilactici strain in a culture medium, (ii) subjecting the inoculated culture medium of the step (i) to conditions suitable for growth of P. acidilactici, and (iii) optionally subjecting the medium resulting from step (ii) to a concentration step.

Non-limiting conditions suitable for the growth of P. acidilactici may be as follows. The bacteria can be cultured on a substrate that may be a liquid broth or solid culture medium, for example in agar plates, using standard culture medium such as Tryptic Soy Agar (TSA), Tryptic Soy Broth (TSB), Man, Rogosa and Sharpe (MRS) agar or broth. Particularly, the culture to be inoculated is in an exponential growth phase close to the stationary phase. Cell multiplication is usually allowed to reach exponential phase, achieving a cell density from 7×10<8> to 2×10<10> colony forming units (CFU)/mL or g substrate. Suitable conditions for the growth of the bacteria are temperatures between 3° and 40° C., for example 35-38° C., for example 37° C., and aerobic conditions or microaerophilic conditions (5% of CO₂). Advantageously, the P. acidilactici described herein grow to high cell densities in industrial scale several culture media, including lactose-free media and other allergen-free media.

Suitable concentration techniques for the cell suspension obtained as described above are known to the skilled person and may include centrifugation or filtration of the culture. The skilled person may apply the concentration technique to a desired extend such that a concentrated suspension in the culture medium may be obtained or, if desired, the culture medium may be completely removed. By centrifuging the culture, for example, at a minimum of 9000 g, cells may be separated from the culture medium (supernatant). Washing steps may be introduced when required. The cell suspension, concentrated cell suspension or cells may then be used directly to obtain the postbiotic preparation or stored.

Optionally, the bacterial cells or cell suspensions obtained by the methods defined above may be subjected to a dehydration process. The dehydration can be carried out by a lyophilization process, but the slurry can also be dried by fluidized bed drying, or by atomization. The cells may be also be directly frozen or stored as cryoballs.

Moreover, the strains of the invention conveniently grow in different culture media to yield cultures with very high cell density (up to 10<10> cfu/ml) in industrial fermenters without losing their activity. All these features are advantageous for industrial handling and commercial use of the strains.

### Compositions

The postbiotic preparations of the first aspect may be used directly for consumption, for example, for oral consumption, or may be used as an ingredient for preparing a product (for example, an oral composition such as a food product or a pharmaceutical composition). Thus, as mentioned above, in a second aspect the invention provides a composition comprising a postbiotic preparation as defined above for use in preventing and/or treating obesity, in reducing body fat, in preventing fat accumulation, or in preventing weight gain. All embodiments described above for the postbiotic preparation of the first aspect also apply to the composition of the first aspect.

While it is possible to administer the probiotic preparation according to the present invention alone (i.e. without any support, diluent or excipient), the postbiotic preparation is typically administered on or in a support as part of a composition. The composition may be, for example, an oral, parenteral, rectal, vaginal, topical, intradermal, transdermal, subcutaneous, sublingual, intravenous or nasopharyngeal composition. In particular embodiments, the composition of the invention comprises a postbiotic preparation of a Pediococcus acidilactici strain selected from the group consisting of CECT 9889, CECT 9967, CECT 9879, and combinations thereof. As mentioned above, this may be alternatively worded as a preparation comprising or consisting essentially of inanimate Pediococcus acidilactici selected from the group consisting of the strains CECT 9889, CECT 9967, CECT 9879, and combinations thereof. In one embodiment, the composition comprises a postbiotic preparation of Pediococcus acidilactici CECT 9889. In other embodiments, the composition comprises a postbiotic preparation of Pediococcus acidilactici CECT 9879. In other embodiments, the composition comprises a postbiotic preparation of Pediococcus acidilactici CECT 9967. In other embodiments, the composition comprises a postbiotic preparation of Pediococcus acidilactici CECT 9889 and Pediococcus acidilactici CECT 9879. In other embodiments, the composition comprises a postbiotic preparation of Pediococcus acidilactici CECT 9889 and Pediococcus acidilactici CECT 9967. In other embodiments, the composition comprises a postbiotic preparation of Pediococcus acidilactici CECT 9879 and Pediococcus acidilactici CECT 9967. In other embodiments, the composition comprises a postbiotic preparation of Pediococcus acidilactici CECT 9889, Pediococcus acidilactici CECT 9879 and Pediococcus acidilactici CECT 9967. Preferably the postbiotic preparation comprised in the composition contains inanimate cells of Pediococcus acidilactici.

In one embodiment, the composition of the second aspect of the invention is an oral composition. In particular embodiments, the oral composition of the invention is selected from the group consisting of a food product, a food ingredient, a food supplement, a pharmaceutical product or a nutraceutical product. These products typically contain additional components (additives) well known to those skilled in the art, such as preservatives, anti-oxidants, emulsifiers, colorants, bulking agents, etc. Appropriate additives are described below. In a very particular embodiment the composition of the second aspect is a food product. Here, the term food is used in a broad sense—and covers food for humans as well as food for animals (i.e. a feed). In a particular embodiment, the food product is for human consumption. In another particular embodiment, the food product is for a non-human animal consumption (feed), in particular for pets or livestock (including fish).

In particular embodiments, the oral composition of the invention is a functional food. In other embodiments the oral composition of the invention is a nutraceutical. In other embodiments the oral composition of the invention is a pharmaceutical composition.

The composition of the present invention may also be used as—or in the preparation of—a pharmaceutical or nutraceutical. Here, the term pharmaceutical or nutraceutical is used in a broad sense- and covers pharmaceuticals or nutraceuticals for humans as well as pharmaceuticals or nutraceuticals for animals (i.e. veterinary applications). In a preferred aspect, the pharmaceutical or nutraceutical is for human use and/or for animal husbandry. When used as—or in the preparation of—a pharmaceutical or nutraceutical composition, the composition of the present invention may be used in conjunction with one or more acceptable excipients or carriers.

The composition of the invention may be presented in any dosage form, for example, solid or liquid, and can be administered by any suitable route. In particular embodiments the pharmaceutical or nutraceutical compositions of the invention are oral compositions (compositions for oral administration). Compositions provided herein suitable for oral administration may be in the form of capsules, cachets, pills, tablets, lozenges, powders, granules, or as a solution or a suspension in an aqueous or non-aqueous liquid, or as an oil-in-water or water-in-oil liquid emulsion, or as an elixir or syrup, or as pastilles.

The tablets, capsules, and other solid dosage forms of the pharmaceutical compositions described herein, such as dragees, pills and granules, may optionally be scored or prepared with coatings and shells, such as enteric coatings and other coatings well known in the art. They may also be formulated so as to provide slow or controlled release of the active ingredient using, for example, hydroxypropylmethyl cellulose in varying proportions to provide the desired release profile, other polymer matrices, liposomes and/or microspheres. Compositions described herein may also be formulated for rapid release, e.g., freeze-dried. These compositions may be formulated to release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Non-limiting examples of acceptable excipients are microcrystalline cellulose, lactose, sodium citrate, calcium carbonate, dibasic calcium phosphate and glycine; disintegrants such as starch, sodium starch glycollate, croscarmelose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc; cryoprotectants, such as DMSO, ethylene glycol, glycerol, 2-Methyl-2,4-pentanediol (MPD), propylene glycol, sucrose and trehalose. Examples of pharmaceutically acceptable carriers include, for example, water, salt solutions, alcohol, silicone, waxes, petroleum jelly, vegetable oils, polyethylene glycols, propylene glycol, liposomes, sugars, gelatin, starch, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like.

The compositions of the invention may also contain a preservative. Examples of preservatives may include antioxidants, chelating agents, antimicrobial preservatives, antifungal preservatives, alcohol preservatives, acidic preservatives, and other preservatives. The compositions of the invention may also contain an antioxidant. Exemplary antioxidants include, but are not limited to, alpha tocopherol, ascorbic acid, ascorbyl palmitate, ascorbyl stearate, ascorbyl oleate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. The compositions of the invention may also contain a chelating agent. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. The compositions of the invention may also contain a buffering agent. Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminium hydroxide, alginic acid, pyrogen-free water, isotonic saline, Ringer's solution, ethyl alcohol, and combinations thereof.

In one embodiment the composition comprises a lubricant and a bulking agent, the composition of the invention comprises a lubricant, for example, magnesium stearate. The amount of lubricant in the composition may be from 0.1 to 5% of the total composition, or from 0.5 to 2% of the total composition or from 0.5 to 1.5% of the total composition. In another embodiment, the composition comprises a bulking agent, for example selected from cellulose, starch and combinations thereof. The cellulose may be for example microcrystalline cellulose. The starch may be for example corn starch. The amount of bulking agent in the composition may be from 30 to 90% of the total composition, or from 40 to 80% of the total composition, or from 50 to 80% of the total composition, or from 30 to 50% of the total composition, or from 40 to 60% of the total composition. In another particular embodiment the composition comprises a cryoprotectant, for example, sucrose, glycerol or trehalose. The amount of cryoprotectant in the composition may be from 0.5 to 20% of the total composition, or from 1 to 10% of the total composition. In a particular embodiment the composition comprises microcrystalline cellulose, starch and magnesium stearate. In a particular embodiment the composition further comprises a cryoprotectant. By “% of the total composition” is understood herein as % by weight when the composition is solid or % by volume when the composition is liquid. In several embodiments the % is understood as % weight of component/weight of total composition.

In some embodiments, the composition comprises a prebiotic. Examples of suitable prebiotics include complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g. guar gum), high amylose cornstarch (HAS), cellulose, b-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, and methylcellulose. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. In particular embodiments the the prebiotic is selected from galacto-oligosaccharide (GOS), fructo-oligosaccharide (FOS), inulin, alginate, xanthan, pectin, locust bean gum (LBG), guar gum, polydextrose (i.e. Litesse®), resistant starch, hydroxypropyl methylcellulose, beta-glucans (derived from example from barely and oat) and arabinoxylan.

In some embodiments the composition comprises a further active ingredient. The further active ingredient may be selected from active ingredients that are effective in preventing and/or treating obesity, in reducing body fat, in preventing fat accumulation, or in preventing weight gain. Non-limiting examples may be selected from the group consisting of Semaglutide, Liraglutide, GLP-1/GIP dual agonists such as tirzepatide, gastrointestinal lipase inhibitor such as Orlistat, appetite suppressants such as phentermine or topiramate, Naltrexone/Bupropion, Setmelanotide, Tirzepatide, fluoxetine, and combinations thereof. In other embodiments, the further active ingredient may be selected from the group consisting of sugar replacers, normoglycemic agents and drugs that control blood sugar levels. In other embodiments the further active ingredient may be one or more different probiotic microorganism, for example, other strains of lactic acid bacteria, including different P. acidilactici strains.

In a particular embodiment, the composition comprises a postbiotic preparation of a Pediococcus acidilactici strain selected from the group consisting of CECT 9889, CECT 9967, CECT 9879, and combinations thereof, in combination with a further active ingredient selected from semaglutide and Orlistat.

### Subjects/Medical Indications

The postbiotic preparation to which the present invention relates is administered to an animal, including for example livestock (including cattle, horses, pigs, sheep, or fish), and humans. In some embodiments the animal is a mammal. In some embodiments the mammal is a companion animal (including pets). In other embodiments the subject is a livestock animal which is not a mammal, for example poultry or fish. In preferred embodiments the subject is a human.

The examples below effectively demonstrate that supplementation with a postbiotic preparation of Pediococcus acidilactici has a beneficial effect in preventing weight gain and improving obesity-related parameters. Therefore, the postbiotic preparation of the invention are for use in preventing and/or treating obesity, in reducing body fat, in preventing fat accumulation, or in preventing weight gain. Preventing and treating obesity in the sense of the present invention encompasses preventing or treating overweight and also preventing or reducing excessive fat accumulation. In one embodiment the postbiotic preparation as defined above is for use in preventing obesity. In another embodiment the postbiotic preparation as defined above is for use in treating obesity. Prophylactic treatment is also contemplated. In another embodiment the postbiotic preparation as defined above is for use in reducing body fat. In another embodiment the postbiotic preparation as defined above is for use in preventing fat accumulation. In another embodiment the postbiotic preparation as defined above is for use in preventing weight gain. In another embodiment the postbiotic preparation as defined above is for use in weight control. In another embodiment the postbiotic composition is for use in reducing appetite (in other words for producing satiety or for reducing food intake).

All embodiments described above for the postbiotic preparation of the first aspect or the compositions of the second aspect apply to the medical uses described in this section.

In one embodiment, the postbiotic preparation of the invention is for administering to subjects that are obese or overweight, or subjects that are at risk of being overweight or obese, for example, genetically predisposed individuals, and/or subjects leading a sedentary life and/or that have a high calory intake. In particular embodiments the postbiotic preparation is for administering to subjects that have a high calory intake, i.e. that ingest a high calory diet or an obesogenic diet. The postbiotic preparation is also useful to subjects that have normal weight who do not want to gain weight for aesthetic reasons. The postbiotic preparation is thus useful for weight control strategies. In another embodiment, the postbiotic preparation is for administering to subjects having normal weight.

In some embodiments, administering the probiotic preparation of the invention lowers the weight of the subject. In particular embodiments, the reduction of weight in the subject is of at least 5% relative to a control. In some embodiments the reduction of weight is of at least 10%, of at least 20%, of at least 30%, or of at least 40%, or of at least 50%, or of at least 55%, or of at least 60%, or of at least 65%, or of at least 70%, or of at least 75%, or of at least 80%, or of at least 85%, or of at least 90%, or of at least 95%, or of at least 100%, relative to a control. In some embodiments the reduction of weight is higher that 100% relative to a control. In some embodiments administering the postbiotic preparation of the invention reduces weight in a subject by 10% to 80%, or by 20% to 100%, or by 40% to 120%, or by 50 to 120%, or by 60 to 120%, or by 70 to 120%, or by 80 to 120%, or by 90 to 120%, or by 100 to 120%, or by 50 to 100%, or by 60 to 100% or by 70 to 100% or by 80 to 100% or by 90 to 100%, with respect to a control. In some embodiments the subject is ingesting an hypercaloric diet. In some embodiments the subject is predisposed to having obesity or overweigth. In some embodiments, administering the postbiotic preparation of the invention reduces gain weight induced by hypercaloric diet in a subject relative to a control. In some embodiments, the control is the subject before administering of the postbiotic preparation of the invention. In some embodiments, the control is another subject that is not administered the postbiotic preparation. In a particular embodiment the subject is predisposed to having obesity or overweight and is ingesting an hypercaloric diet and the control is the same subject before administration of the postbiotic preparation of the invention or another subject predisposed to obesity or overweight that is ingesting the same hypercaloric diet and is not being administered the postbiotic preparation of the invention.

In some embodiments, the effect in preventing and/or treating obesity, in reducing body fat, in preventing fat accumulation, or in preventing weight gain, or in reducing food intake and/or appetite is achieved by administering the postbiotic preparation of the invention during at least 2 weeks. In other embodiments the postbiotic preparation is administered during at least 4 weeks. In other embodiments the postbiotic preparation is administered during at least 8 weeks. In other embodiments the postbiotic preparation is administered during at least 12 weeks. In other embodiments the postbiotic preparation is administered for at least 6 months. In certain embodiments the postbiotic preparation is administered indefinitely.

While preventing and/or treating obesity (or by preventing excessive fat accumulation, or by preventing weight gain or by reducing body fat), the postbiotic preparation may reduce the risk of suffering from metabolic disorders that are associated with overweight, obesity and/or excessive fat accumulation. Metabolic disorders that are associated with overweight, obesity and/or excessive fat accumulation are well known to skilled artisans. For example, these disorders include cardiovascular diseases such as coronary heart disease; insulin resistance; hypertension; sleep apnoea, respiratory problems and/or dyslipidemia. In particular, embodiments of the invention the metabolic disorders that are associated with obesity and/or excessive fat accumulation are selected from coronary heart disease, insulin resistance, hypertension, dyslipidaemia, and sleep apnoea.

It is also relevant that the postbiotic preparation of the invention reduced serum makers of liver disease (ALT) as well as fat accumulation in the liver tissue (see examples below). This translates in preventing or reducing steatosis and in preventing liver damage. Thus, in particular embodiments, the postbiotic preparation of the invention is for use in preventing and/or treating steatosis. In other embodiments, the postbiotic preparation is for use in the prevention of liver damage.

The postbiotic preparation of the invention may be used in combination with a further active ingredient. Thus, in one embodiment the invention refers to a postbiotic composition as defined above for use in preventing and/or treating obesity, in reducing body fat, in preventing fat accumulation, or in preventing weight gain, when used in combination with a further active ingredient. This embodiment can be reworded as the use of a postbiotic composition as defined above for the preparation of a medicament for preventing and/or treating obesity, in reducing body fat, in preventing fat accumulation, or in preventing weight gain, in combination with a further active ingredient. The invention also contemplates a method for treating. This aspect can also be formulated as a method for preventing and/or treating obesity, in reducing body fat, in preventing fat accumulation, or in preventing weight gain, the method comprising administering an a postbiotic preparation as defined above, in combination with a further active ingredient.

The further active ingredient is selected, in particular embodiments, from active ingredients that are effective in preventing and/or treating obesity, in reducing body fat, in preventing fat accumulation, or in preventing weight gain. Active ingredients appropriate for use in combination with the postbiotic preparation may be selected from the group consisting of Semaglutide, Liraglutide, GLP-1/GIP dual agonists such as tirzepatide, gastrointestinal lipase inhibitor such as Orlistat, appetite suppressants such as phentermine or topiramate, Naltrexone/Bupropion, Setmelanotide, Tirzepatide, fluoxetine and combinations thereof. The use of the postbiotic preparation in combination with these drugs allows for reducing the dose of the drug, thus also limiting their side effects. In a particular embodiment the further active ingredient appropriate for use in combination with the postbiotic preparation may be selected from the group consisting of liraglutide, naltrexone-bupropion, obesity, orlistat, phentermine, phentermine-topiramate, semaglutide, setmelanotide, tirzepatide. In another embodiment the further active ingredient appropriate for use in combination with the postbiotic preparation may be selected from the group consisting of Glucomannan or other viscous fibers, Gymnema sylvestre, Griffonia simplicifolia (5-HTP), Caralluma fimbriata, Conjugated linoleic acid, Garcinia cambogia, Flaxseed, Psyllium Husk, Green Tea, Yerba mate, Chili Peppers, Coffee, Fenugreek, and combinations thereof.

The further active ingredient may also be another bacterial strain, in particular, a further bacterial strain that is effective in preventing and/or treating obesity, in reducing body fat, in preventing fat accumulation, or in preventing weight gain.

The postbiotic preparation or composition and the further active ingredient are administered concomitantly or separately, in any order, within a therapeutically effective interval.

The postbiotic preparation of the invention may also be conveniently used in combination with a low-calorie diets and/or regular exercise. Thus, one embodiment the invention refers to a postbiotic composition as defined above for use in preventing and/or treating obesity, in reducing body fat, in preventing fat accumulation, or in preventing weight gain, when used in combination with a low-calorie diets and/or regular exercise. This embodiment can be reworded as the use of a postbiotic composition as defined above for the preparation of a medicament for preventing and/or treating obesity, in reducing body fat, in preventing fat accumulation, or in preventing weight gain, in combination a low-calorie diets and/or regular exercise. The invention also contemplates a method for treating. This aspect can also be formulated as a method for preventing and/or treating obesity, in reducing body fat, in preventing fat accumulation, or in preventing weight gain, the method comprising administering an a postbiotic preparation as defined above, in combination with a low-calorie diets and/or regular exercise.

### Dosage

In one embodiment, the postbiotic preparation in accordance with the present invention is administered at a dose comprising from 10⁷ to 10¹² cells of P. acidilactici per day per animal. In one embodiment, the dose of postbiotic preparation comprises from 10⁸ to 10¹¹ cells of P. acidilactici per day per animal, in particular, from 10⁹ to 10¹¹ cells of P. acidilactici per day per animal, for example, 10⁹, 10¹⁰, or 10¹¹ cells of P. acidilactici per day per animal. The cells are inanimate cells and usually refer to the total amount of P. acidilactici cells, i.e. if the postbiotic preparation contains P. acidilactici CECT 9879, CECT 9889, and CECT 9967 strains, the dose of, for example, 10¹⁰ cells of P. acidilactici per day per animal, refers to the sum of P. acidilactici CECT 9879, CECT 9889, and CECT 9967 cells.

When the bacteria, such as a strain of Pediococcus acidilactici, for example Pediococcus acidilactici CECT 9889, CECT 9967 or CECT 9879, are used in the present invention together with other bacterial strains, the bacteria may be present in any ratio capable of achieving the desired effects of the invention described herein. Typically, the Pediococcus acidilactici to each other or other species ratio is in the range 1:100 to 100:1, suitably 1:50 to 50:1, particularly 1:20 to 20:1, more particularly 1:10 to 10:1, still more particularly 1:5 to 5:1, yet more particularly 1:3 to 3:1 and even more particularly 1:2 to 2:1 and most particularly 1:1.5 to 1.5:1.

For completeness the invention is further described in the following numbered embodiments:

1. A postbiotic preparation of Pediococcus acidilactici (P. acidilactici) for use in preventing and/or treating obesity, in reducing body fat, in preventing fat accumulation, in reducing appetite, in reducing food intake, or in preventing weight gain.

2. A postbiotic preparation of P. acidilactici for use in preventing and/or treating steatosis or for preventing liver damage.

3. The postbiotic preparation for use according to any one of the preceding embodiments, comprising or consisting essentially of inanimate P. acidilactici cells.

4. The postbiotic preparation for use according to any one of the preceding embodiments, comprising a lysate of P. acidilactici and/or a culture supernatant of P. acidilactici.

5. The postbiotic preparation for use according to any one of the preceding embodiments, wherein the P. acidilactici is selected from the group consisting of P. acidilactici deposited at the “Colección Española de Cultivos Tipo” (CECT) with identification reference CECT 9879, P. acidilactici deposited at the “Colección Española de Cultivos Tipo” (CECT) with identification reference CECT 9889, P. acidilactici deposited at the “Colección Española de Cultivos Tipo” (CECT) with identification reference CECT 9967, and combinations thereof.

6. The postbiotic preparation for use according to any one of the preceding embodiments, wherein the use comprises administering to an animal in need thereof from 10⁷ to 10¹¹ cells of P. acidilactici per day per animal, in particular, from 10⁹ to 10¹¹ cells of P. acidilactici per day per animal.

7. The postbiotic preparation for use according to any one of the preceding embodiments, wherein the use comprises administering to an animal in need thereof around 10¹⁰ cells of P. acidilactici per day per animal.

8. A composition comprising a postbiotic preparation of P. acidilactici as defined in any one of embodiments 1-7, for use in preventing and/or treating obesity, in reducing body fat, in preventing fat accumulation, in reducing appetite, in reducing food intake, or in preventing weight gain.

9. A composition comprising a postbiotic preparation of P. acidilactici as defined in any one of embodiments 1-7, for use in preventing and/or treating steatosis or for preventing liver damage.

10. The composition for use according to any one of embodiments 8-9, wherein the composition is an oral composition.

11. The composition for use according to the preceding embodiment, wherein the oral composition is a food or feed product, a food or feed ingredient, or a food or feed supplement.

12. The composition for use according to the preceding embodiment, wherein the food or feed product is a functional food or feed product, and optionally further comprises food or feed additives selected from the group consisting of preservatives, anti-oxidants, emulsifiers, colorants, and bulking agents.

13. The composition for use according to any one of embodiments 8-9, wherein the composition is a pharmaceutical or nutraceutical composition and also comprises pharmaceutically acceptable excipients and carriers.

14. The composition for use according to the preceding embodiment, comprising a lubricating agent, and/or a bulking agent, and/or an enteric coating.

15. The postbiotic preparation for use according to any one of embodiments 1-7 or the composition for use according to any one of claims 8-14, that is administered to subjects consuming a hypercaloric diet.

16. The postbiotic preparation for use according to any one of embodiments 1-7 or 15 or the composition for use according to any one of claim 8-14 or 15, that is administered to an animal.

17. The postbiotic preparation or the composition for use according to the preceding embodiment, wherein the animal is selected from a human, a pet, or life-stock (including cattle and fish).

18. The postbiotic preparation for use according to any one of embodiments 1-7 or 15-17 or the composition for use according to any one of claim 8-14 or 15-17, when used in combination with (a) a further active ingredient that is effective in preventing and/or treating obesity, in reducing body fat, in preventing fat accumulation, in reducing appetite, in reducing food intake, in preventing adipogenesis in the adipose tissue, or in preventing weight gain.

19. The postbiotic preparation or composition for use according to the preceding embodiment, wherein the further active ingredient is selected from the group consisting of a probiotic microorganism, including P. acidilactici strains, Semaglutide, Liraglutide, GLP-1/GIP dual agonists such as tirzepatide, gastrointestinal lipase inhibitor such as Orlistat, appetite suppressants such as phentermine, topiramate, or natural appetite suppressants, Naltrexone/Bupropion, Setmelanotide, Tirzepatide, and combinations thereof.

20. The postbiotic preparation or composition for use according to any one of embodiments 18-19, wherein the postbiotic preparation or composition and the further active ingredient are administered separately, in any order, within a therapeutically effective interval.

21. The postbiotic preparation for use according to any one of embodiments 1-7 or 15-17 or the composition for use according to any one of claim 8-14 or 15-17, when used in combination with, and/or (b) a low-calorie diets and/or regular exercise.

22. The postbiotic preparation for use according to any one of embodiments 1-7 or 15-21 or the composition for use according to any one of claim 8-14 or 15-21, comprising P. acidilactici CECT 9879.

23. The postbiotic preparation for use according to any one of embodiments 1-7 or 15-21 or the composition for use according to any one of claim 8-14 or 15-21, comprising P. acidilactici CECT 9889.

24. The postbiotic preparation for use according to any one of embodiments 1-7 or 15-21 or the composition for use according to any one of claim 8-14 or 15-21, comprising P. acidilactici CECT 9967.

25. The postbiotic preparation for use according to any one of embodiments 1-7 or 15-21 or the composition for use according to any one of claim 8-14 or 15-21, comprising P. acidilactici CECT 9879, P. acidilactici CECT 9889, and P. acidilactici CECT 9967.

26. A postbiotic preparation for use according to any one of the preceding embodiments, wherein the postbiotic preparation is obtainable by submitting P. acidilactici cells to a treatment to kill or inactivate said cells.

27. A postbiotic preparation for use according to the preceding embodiment wherein the treatment to kill or inactivate the cells comprises a treatment selected from the group consisting of thermal inactivation, such as pasteurization, sterilization, or ohmic heating, pulsed electric fields, ultrasound, irradiation, supercritical carbon dioxide treatment, phenolization, formalin treatment, and formaldehyde treatment.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1: C. Elegans Model

### 1. Materials and Methods

### 1.1. Strains and Culture

The postbiotic pA1c®HI Pediococcus acidilactici CECT 9879, postbiotic pA2cHI Pediococcus acidilactici CECT 9889 and probiotic pA1c® Pediococcus acidilactici CECT 9879, were used at a concentration of 5×10⁶ CFU/mL and 5×10⁶ cells/mL respectively for all the assays. All postbiotic preparations were obtained by heat-inactivation of the corresponding P. acidilactici strains. The Caenorhabditis elegans (C. elegans) strains used were: N2 Bristol as wild-type strain, and the daf-16: GFP mutant strain. All strains were obtained from the Caenorhabditis Genetics Center (CGC, University of Minnesota, Minneapolis, MN, USA).

C. elegans was cultured on nematode-growth-medium (NGM) at 20° C. Escherichia coli OP50 (E. coli, grown in LB Broth Lennox at 37° C.) was utilized as standard nematode sustenance.

### 1.2. Experimental Design

All tests were carried out in quadruplicate, in six-well cell culture plates with 4 mL NGM or with 4 mL glucose-loading (10 mM) NGM (NGMg) per well (with the pA1c®, the pA1c®HI and CECT 9889 pA2cHI spread or not inside the medium). Glucose was added to the medium with the aim of mimicking hypercaloric and obese conditions (Yavorov-Dayliev et al., 2023).

Plates with Orlistat (1.5 mg/mL, Sigma Aldrich, St. Louis, MO, USA) were used as a positive control of fat accumulation reduction based on previous studies in where this drug for human use (pancreatic lipase inhibitor) was also used as positive control. All experiments were performed at the concentration of 5×10⁶ CFU/mL (colony-forming unit/millilitre of water) of pA1c®HI, pA2cHI and pA1c®. After pA1c®HI, pA2cHI and pA1c® were added to NGM, plates were allowed to solidify and dry in the dark to protect them from light oxidation.

Thereafter, 150 μL of an overnight culture of E. coli OP50 were seeded, and plates were again incubated until dried at room temperature in the dark. For all assays, gravid animals were subjected to a standard hypochlorite treatment to obtain age-synchronized worms (wild-type or mutants). The eggs were allowed to hatch overnight in M9 medium, and about 2000 L1 larvae were transferred onto plates and grown until L4 or one-day adult stage.

### 1.3. Nile Red Staining

Nile Red staining is a dye for neutral lipids and is useful for the quantification of the fat accumulation of the worm. Briefly, L4 worms grown in NGM or NGMg with different treatments (control, orlistat, pA1c® HI, pA2cHI and pA1c®,) were collected in 1.5 mL tubes and washed three times with PBST (0.01% of Triton X-100 in phosphate-buffered saline). Then, the worms were put on ice for 15 min and fixed in 40% isopropanol for 3 min. Staining was carried out by adding 150 μL of Nile Red solution (3 ug/mL) per tube and incubating (30 min) with moderate shaking at room temperature in the dark. After that, the worms were washed in PBST and mounted on a 2% agarose pad for microscopy visualization.

### 1.4. Localization of Daf-16 Gene

Aged-synchronized L1 larvae were transferred to NGM or NGMg plates previously supplemented with or without pA1c®HI and pA1c®, and incubated for 46 h (until they reached L4 larval stage) at 20° C. Then, worms were mounted on 2% agarose pads with 1% sodium azide to anesthetize the worms.

### 1.5. Image Acquisition and Quantification

For all conditions tested, approximately 300 animals were fixed and stained. Images of Nile Red and daf-16: GFP assays were taken under the same conditions (calibration: 0.68 μm/px; optics: SHR Plan Apo 1×; numerical aperture: 0.15; refractive index: 1; format: 1280×960 2×2 Binning; exposure: ME 800 ms (−2.0 EV); Analog Gain: 2.00; metering mode: average; BithDepth: 8; zoom: 10×, Fluorescent images of Nile Red stained worms were captured at 10× magnification on a Nikon SMZ18 research stereomicroscope equipped with an epi-fluorescence system and a DS-FI1C refrigerated color digital camera (Nikon Instruments Inc., Tokyo, Japan).

Images were taken at the same conditions and integration time under a GFP filter (Ex 480-500; DM 505; BA 535-550). Regarding the daf-16 intracellular localization assay, worms were photomicrographed using a GFP (with excitation of 340-380 nm and emission of 435-485 nm) in a Nikon SMZ18 (Nikon Instruments Inc., Tokyo, Japan) fluorescence microscope.

In all cases, the image analysis of the Nile Red and of the daf-16: GFP assays was performed using ImageJ v1.53e software. The mean value, calculated as the fluorescence mean value per pixel, together with the integrated density and the volume of the worms, were determined. Approximately 25-40 worms were examined in four independent experiments for each condition.

### 1.6. Egg Lying and Development

To ensure that the supplementation with the bacterium did not affect the development of the worm, egg lying was observed in young adult nematodes (day 3 of growth) grown on NGMg agar plates supplemented or not with pA1c® HI and pA1c®. The images were taken directly on the plates at 135× magnification using a Nikon SMZ18 stereomicroscope equipped with a Nikon DS-Fi1C high-definition colour camera (Nikon Instruments Inc., Tokyo, Japan).

### 1.7. Statistical Analysis

C. elegans body fat reduction (Nile Red) between supplemented groups and control condition, together with daf-16: GFP assay, were evaluated by the one-way ANOVA test followed by the Student-Newman-Keuls (SNK) multiple comparisons test when statistical significance (p<0.05) was reached in the ANOVA test. All tests were performed using StataSE v14 software (StataCorp LLC, College Station, TX, USA).

### 2. Results

2.1. The Postbiotic Pediococcus acidilactici CECT 9879 pA1c®HI Reduces Fat Accumulation in C. elegans with Efficacy Values Comparable to Those of Certain Anti-Obesity Drugs Such as Orlistat

C. elegans, a free-living nematode, is an experimental in vivo model that has been extensively employed in the study of obesity due to its favourable features such as its compact size, short life cycle, large brood size, easy handling, low cost, availability of complete genetic information, 65% conserved human diseases-associated genes and relatively easy genetic manipulation (Yue et al., 2021). The measurement of fat storage and lipid droplets in C. elegans is possible by the visualization under microscopy of fat-soluble dyes such as Nile Red (Escorcia et al., 2018).

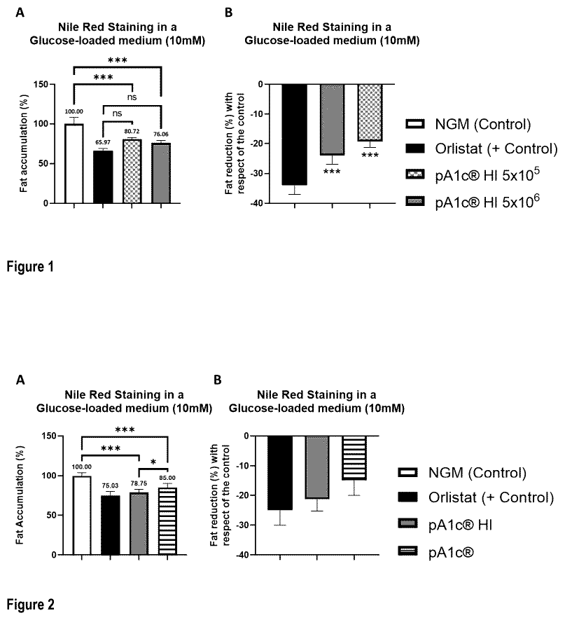

In a glucose-loading NGM medium similar to a high-glucose diet (HGD) associated with obesity, pA1c®HI significantly (p<0.001) reduced fat accumulation in wild-type C. elegans (25% and 20% of fat reduction, at a dose of 5×10⁶ cells/mL and 5×10⁵ cells/mL respectively) with respect to control worms (FIG. 1A, B). Moreover, there were no significant differences between pA1c®HI and the positive control (anti-obesity drug Orlistat), highlighting the strong fat-reducing activity of the natural postbiotic pA1c®HI (more effective at 5×10⁶ cells/ml dose).

Furthermore, in another independent experiment, pA1c®HI (5×10⁶ CFU/mL) significantly (p<0.001) reduced fat accumulation by 20-25% in a glucose-loaded (10 mM) NGM medium compared with control worms, while pA1c®-supplemented worms exhibited a 15% of fat reduction (FIG. 2A, B). Therefore, the anti-obesogenic activity of pA1c®HI was corroborated, and it was demonstrated that pA1c®HI is more effective (p<0.05) than the pA1c® in reducing C. elegans lipid accumulation (at the same dose, 5×10⁶ CFU/mL). These results confirm that the efficacy values of the postbiotic pA1c®HI are comparable to those of certain anti-obesity drugs such as Orlistat, highlighting the strong fat-reducing activity of pA1c®HI.

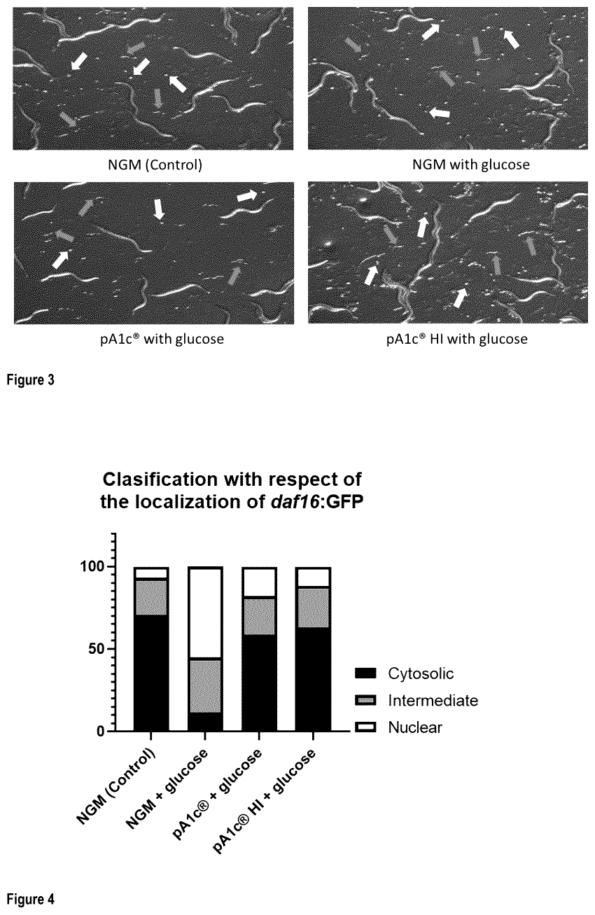

Moreover, the fat-reducing activity of pA1c®HI did not present any detrimental effect on the organism's growth and correct development. Comparing pA1c®HI-supplemented worms with control and pA1c®-supplemented worms, in plates of NGM medium with and without glucose, it was observed that the worms of the pA1c®HI group exhibited the presence of eggs (white arrows) and L1 larvae (grey arrows) as expected, with no differences in their time of appearance (FIG. 3). This shows that pA1c®HI is a safe product and that the lipid accumulation reduction is not accompanied by toxicity or harmful effects on the organism's growth and development.

2.2. The Postbiotic Pediococcus acidilactici CECT 9879 pA1c®HI Counteracted the Nuclear Translocation of Daf-16 Under Hyperglycemic Conditions Confirming the Reducing Effect of pA1c®HI on Fat Accumulation.

It is well known that the gene daf-16 is one of the most important genes of the lipid metabolism of C. elegans and governs the fat accumulation (Xu et al., 2019). Moreover, a high-glucose diet (HGD), is associated with obesity. In addition to mimicking hypercaloric and obese conditions, previous studies considered HGD as a factor to develop oxidative stress (Buranasin et al., 2018). daf-16 nuclear localization depends on many factors, including exposure to stress. Therefore, contemplating HGD as an environmental factor that causes stress to the worms, we hypothesized that daf-16 would transpose to the nucleus in response to these stress conditions, such as the presence of glucose in the medium.

Thus, while a higher cytosolic expression of daf-16 was observed in control worms grown in NGM plates, a nuclear-daf-16 translocation was observed when glucose (10 mM) was added into the medium, confirming our hypothesis. However, pA1c®HI- and pA1c®-supplemented worms reverted the glucose-nuclear-localization of daf-16 (FIG. 4). These findings would confirm the reducing effect of pA1c® and pA1c®HI on fat accumulation and demonstrate that pA1c®HI is more effective than pA1c®.

2.3. The Postbiotic Pediococcus acidilactici CECT 9889 pA2c HI Reduce the Lipid Accumulation of C. elegans

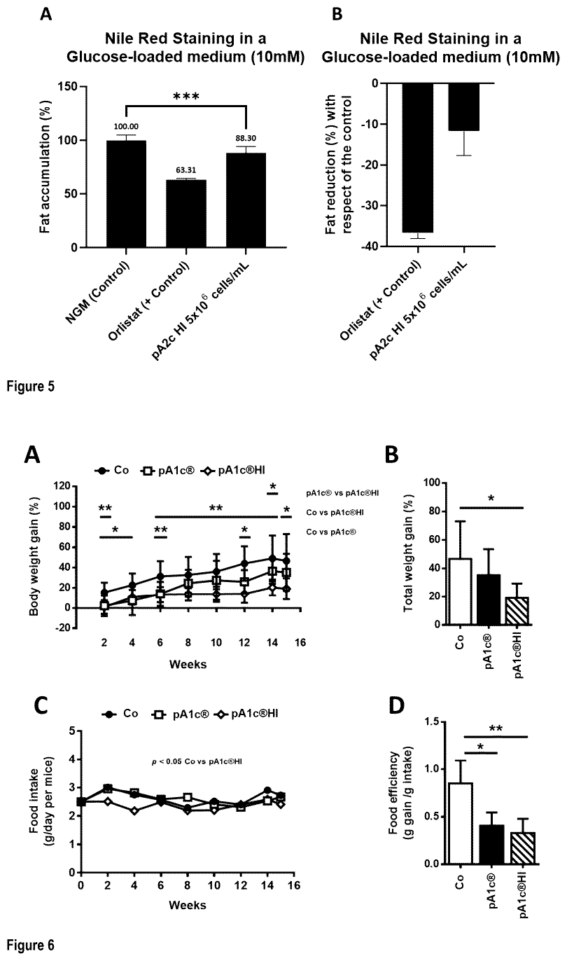

The postbiotic Pediococcus acidilactici CECT 9879 pA2cHI at a dose of 5×10⁶ cells/mL significantly (p<0.001) reduced lipid storage (12%) in wild-type C. elegans in a glucose-loading NGM medium with respect to control worms (FIG. 5A, B)

In conclusion, we have shown that the strains pA1c®HI and pA2cHI reduce (p<0.001) fat accumulation in C. elegans. Moreover, we have demonstrated that the molecular mechanism by which pA1c®HI exerts this fat-reducing effect is modulating the daf-16 localization under a high-glucose diet (reverting the daf-16-nuclear-translocation effect of high-glucose and returning daf-16 into the cytosol).

Taken together, our data suggest that supplementation with 2 strains of Pediococcus acidilactici inactivated as postbiotics (CECT9879 and CECT9889, pA1c®HI and pA2cHI respectively) significantly improve the response of the lipid metabolism in C. elegans. Hence, pA1c®HI and pA2HI could be considered as potential postbiotic strains for the prevention and treatment of obesity-related conditions.

### Example 2: Murine Model

### 1. Materials and Methods

### 1.1. Experimental Design

Thirty male C57BL/6 mice (Charles River Laboratories) aged 9 weeks were acclimated for 2 weeks with a standard chow diet. The animals were randomly divided and allocated into three groups (n=10 each): (1) Control group (Co), animals receiving a high-fat diet (HFD); (2) animals receiving HFD plus a probiotic formulation (pA1c® Pediococcus acidilactici CECT 9879, prepared as explained below), and (3) animals receiving HFD plus a postbiotic formulation with heat-inactivated (HI) (pA1c®)-HI Pediococcus acidilactici CECT 9879, prepared as explained below).

During the study, all groups had ad libitum access to the diets and water. To verify food acceptance, food and drink intake were visually confirmed by one researcher throughout the experimental study. Food intake was monitored every 2 weeks. At the end of the study (15 weeks), all animals were sacrificed by cervical dislocation for the collection tissue samples (liver, adipose tissue and colon) and blood. Tissue samples for quantitative real-time PCR were cut and frozen at −80° C. immediately after collection. Tissue samples for histological analysis were washed with PBS and fixed in a formaldehyde solution (10%) for 24 h. Blood samples were centrifuged for 8 min at 2000 rpm, and serum samples were stored at −80° C. until analysis. Stool samples were collected at the end of the study for the analysis of the faecal microbiota through 16S rRNA sequencing. All animal procedures were performed under protocols approved by the Institutional Committee on Care and Use of Laboratory Animals (CEEA, University of Navarra) (Protocol number: CEEA/017-20).

### 1.2. Diets and Postbiotic Preparation

The probiotics/postbiotics were mixed with the HFD every 2 weeks under sterile conditions and kept at 4° C. until use. The HFD (TD.06414, Envigo, Tekla, USA) contains 60% of kcal from fats (detailed information in table 1).

TABLE 1
Nutritional information TD.06414 (Envigo)

| Formula | g/Kg | Composition | % by weight | % kcal from |
|---|---|---|---|---|
| Lard | 310.0 | Fat | 34.3 | 60.3 |
| Casein | 265.0 | Carbohydrate | 27.3 | 21.4 |
| Maltodextrin | 160.0 | Protein | 23.5 | 18.3 |
| Sucrose | 90.0 | Kcal/g | 5.1 | |
| Cellulose | 65.5 | | | |
| Mineral Mix, AIN-93G-MX (94046) | 48.0 | | | |
| Soybean Oil | 30.0 | | | |
| Vitamin Mix, AIN-93-VX (94047) | 21.0 | | | |
| L-Cystine | 4.0 | | | |
| Calcium Phosphate, dibasic | 3.4 | | | |
| Choline Bitartrate | 3.0 | | | |
| Blue Food Color | 0.1 | | | |

Information obtained from Envigo Teklad Diets

The dose of the pA1c® in the diet was adjusted to 1×10¹⁰ CFU of Pediococcus acidilactici CECT9879 per day/animal, and the microbiological load in the diet was confirmed using the classic plate count method (37° C. and 5% CO₂ during 48 h). Before the study, pA1c® survival in HFD was determined to confirm that animals received viable microorganisms during the intervention.

The dose of the pA1c®HI in the diet was adjusted to cells of inactivated Pediococcus acidilactici CECT9879 per day/animal. Before the study, pA1c®HI counting in HFD was determined to confirm that the animals received the total cells of Pediococcus acidilactici during the intervention.

### 1.3. Body Weight, Food Efficiency and Fasting Blood Glucose

Body weight (BW) was recorded every 2 weeks. Food efficiency was determined as grams of weight gain/grams of food intake between the control and treated animals. 12 h fasting blood glucose (FBG) was determined every 2 weeks using a glucometer (Accu-chek Aviva, Roche, Basel, Switzerland), and blood samples were obtained from the tip of the tail vein.

1.4. Histological analysis Fixed tissue samples (liver and adipose tissue) were embedded in paraffin and cut into 3 μm thick sections. Sections were stained with hematoxylin and eosin (H&E) for light microscopy examination. Detailed information of the protocol used for the preparation of the tissue samples and the immunolabeling is described in Cabello-Olmo et al (Nutrients 2022, 14).

The liver slides were digitized using a histology slide scanner Aperio CS, running under the Scan Scope Console software (v.10.2.0.2352, Leica Biosystems) and further analysed using Fiji software. In H&E stained liver sections, we analysed steatosis (expressed as a percentage of the area occupied by lipid droplets) in ten random images (20×) for each animal (n=8 for each group). In colon sections, we measured the percentage of GLP-1 positive cells per total cells in ten random fields (20×) for each animal (n=4-7 for each group). In the adipose tissue sections, we evaluated adipocytes area and diameter with the Adiposoft software. We analysed digitalized images of H&E stained adipose sections for each animal (n=8 for each group).

### 1.5. Quantitative Real-Time PCR (RT-qPCR)

Frozen (−80° C.) liver and adipose tissues were used for total RNA extraction using RNeasy Mini Kit (Qiagen) according to the manufacturer's instructions. The purity and concentration were determined by spectrophotometry using Nanodrop One (Thermo Scientific). Then, cDNA synthesis (RT) was performed using Superscript™ IV VILOTMRT Premix with enzDNase™ (Invitrogen) following the manufacturer's instructions. The mRNA expression levels of hepatic glycogenesis-related genes (glucose-6-phosphatase (G6Pase), phosphoenolpyruvate carboxykinase (Pepck), and glucokinase (Gck)) and lipid metabolism-related genes (sterol regulatory element-binding protein (Srebp), fatty acid synthase (Fasn), peroxisome proliferator-activated receptor a (Ppara), peroxisome proliferator-activated receptor γ (Pparγ), acetyl-coenzyme A oxidase 1 (Acox), carnitine palmitoyl-transferase 1 (Cpt1), and CD36) were analysed and further normalized using H3 or Gapdh as a house-keeping gene for liver or adipose tissue respectively. Quantitative real-time PCRs (qPCR) were performed with the IQ SYBR Green Supermix (Bio-Rad) in a CFX96 Real-Time System (Bio-Rad). Data is expressed as the relative mRNA normalized to H3 or Gapdh and analysed according to the comparative cycle threshold method (2^{-ΔΔCT}). Primer sequences for the targeted mouse genes and sources are shown in Table 2.

TABLE 2
Primer sequences

| | Forward/ | | |
|---|---|---|---|
| Target gene | Reverse | Primer sequence (5′ to 3′) | SEQ ID NO |
| Acox | F | CTATGGGATCAGCCAGAAAG | SEQ ID NO: 1 |
| | R | AGTCAAAGGCATCCACCAA | SEQ ID NO: 2 |
| CD36 | F | CACAGCTGCCTTCTGAAATGTGTGG | SEQ ID NO: 3 |
| | R | TTTCTACGTGGCCCGGTTCTAATTC | SEQ ID NO: 4 |
| Cpt1 | F | TCTAGGCAATGCCGTTCAC | SEQ ID NO: 5 |
| | R | GAGCACATGGGCACCATAC | SEQ ID NO: 6 |
| Fasn | F | AGCCATGGAGGAGGTGGTGAT | SEQ ID NO: 7 |
| | R | GTGTGCCTGCTTGGGGTGGAC | SEQ ID NO: 8 |
| H3 | F | AAAGCCGCTCGCAAGAGTGCG | SEQ ID NO: 9 |
| | R | ACTTGCCTCCTGCAAAGCAC | SEQ ID NO: 10 |
| Pparα | F | ACAAGGCCTCAGGGTACCA | SEQ ID NO: 11 |
| | R | GCCGAAAGAAGCCCTTACAG | SEQ ID NO: 12 |
| Pparγ | F | GCTGTTATGGGTGAAACTCTG | SEQ ID NO: 13 |
| | R | GAATAATAAGGTGGAGATGCAGG | SEQ ID NO: 14 |
| Pepck | F | AGTCATCATCACCCAAGAGC | SEQ ID NO: 15 |
| | R | GGGATGACATACATGGTGC | SEQ ID NO: 16 |
| Srepb | F | CACTTCATCAAGGCAGACTC | SEQ ID NO: 17 |
| | R | CGGTAGCGCTTCTCAATGGC | SEQ ID NO: 18 |

### 1.6. Statistical Analysis

Data are presented as mean±standard deviation (SD). All statistical procedures were performed using GraphPad Prism 8.0.1 software. Statistical significance was set at p<0.05. * denotes p<0.05, ** denotes p<0.01, *** denotes p<0.001.

Data were analysed by using one-way analysis of variance (ANOVA) followed by Tukey's post-hoc test or the Kruskal-Wallis test followed by the Dunn's test for multiple comparisons, as appropriate.

### 2. Results

2.1. The Postbiotic Pediococcus acidilactici CECT 9879 pA1c®HI Prevents BW Gain and Improved Obesity-Related Parameters

After 15 weeks with High Fat Diet like an hypercaloric conditions in obesity, the Control group (Co) increased the body weight (BW) by 46.7±26.4%. However, the supplementation with the postbiotic Pediococcus acidilactici CECT 9879 pA1c®HI led to a rise in BW of only 19.1±10.3%. The animals supplemented with the postbiotic gained 40% less weight than the non-supplemented animals (FIG. 6A). Moreover, FIG. 6B shows that the total weight gained (%) after 15 weeks, was significantly lower (p<0.05) with pA1c®HI vs. the Co group, evidencing the strong anti-obesogenic effect of pA1c®HI.

Furthermore, statistically significant differences were also observed between the pA1c®HI and pA1c® groups (p<0.05) (FIGS. 6A and 6B), emphasising that pA1c®HI is more effective in reducing total BW gain and modulating the lipid metabolism than pA1c®.

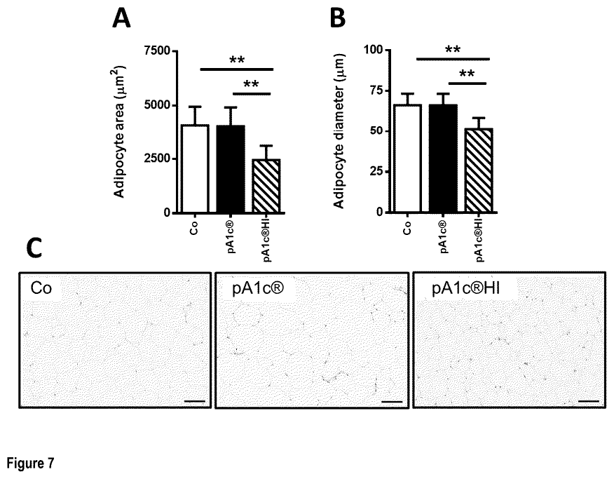

Mice ate significantly less amount of feed when the postbiotic pA1c®HI was supplemented in the diet vs the Co group (p<0.05) (FIG. 6C), suggesting that pA1c®HI could affect the satiety centre of the animal by activating it. Besides, as shown in FIG. 6D, we found differences in food efficiency (described as grams of weight gain/grams of food intake) between the control and treated animals (Co vs pA1c®, p<0.05; Co vs pA1c®HI, p<0.01). Therefore, this confirmed that BW control was related to postbiotic administration rather than variations in food intake. We analysed the area and diameter of adipocytes in adipose tissue and found that, the administration of the postbiotic pA1c®HI slowed the increase in adipocyte size (p<0.01) (FIGS. 7A-7B). It is directly related to a lower amount of adipose tissue in the pA1c®HI group (FIG. 7C) and directly correlated to the lower BW gain, demonstrating once again the adipose tissue-reducing activity of pA1c®HI.

2.2. pA1c®HI Controlled Glucose Dysregulation in HFD-Fed Mice

High glucose levels and insulin resistance are crucial factors in the development of obesity. Insulin, which is responsible for regulating blood sugar, is hindered by resistance, raising glucose levels, and promoting fat accumulation. This interaction highlights the need to comprehensively address these metabolic imbalances to effectively prevent and treat obesity.

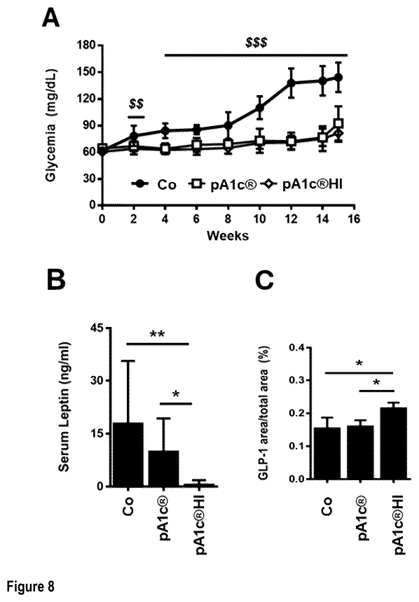

At the beginning of the study, blood glucose levels were similar between the groups (average FBG 62.4+3.9 mg/dL, range 54.0-70.0 mg/dL). After starting the administration of the diets, FBG began to increase and continued ascending in the Co group. However, mice in the pA1c®HI group, remained blood glucose levels constant for 15 weeks. The blood glucose levels were similar to those observed at the beginning of the study. Similar behaviour was observed in pA1c®HI group. We observed significant differences in glucose levels after 2 weeks of supplementation with pA1c®HI with respect to the Co group (p<0.05 week 2 and p<0.01 from week 4 to the end of the study). No differences were found between pA1c® and pA1c®HI groups in FBG levels during the 15 weeks of the study (p>0.05) (FIG. 8A).

Leptin, a hormone produced by fat cells, is pivotal in the development of obesity.

It regulates appetite and energy balance by sending signals to the brain about the amount of fat stored. In obesity, elevated leptin levels may indicate resistance, reducing the response to satiety signals. This resistance contributes to increased intake and excess fat storage, thereby contributing to the perpetuation of obesity.

Furthermore, serum leptin levels of the pA1c®HI group were strongly inhibited when comparing to control group (pA1c®HI: 0.5±1.3 ng/ml; p<0.01) and between pA1c®HI group and pA1c® group (pA1c®: 9.9+9.4 ng/ml and pA1c®HI: 0.5±1.3 ng/ml; p<0.05). This indicates that the postbiotic supplementation affect satiety and the food intake, and prevent the development of obesity and demonstrating that pA1c®HI is more effective than pA1c® in the management and treatment of obesity (FIG. 8B).

On the other hand, the immunohistochemical quantification of colonic GLP-1 revealed an increased GLP-1⁺ area in the pA1c®HI group with respect to the other two groups (Co: 0.15±0.03%, pA1c®: 0.16±0.02% and pA1c®HI: 0.22±0.02%; p<0.05) (FIG. 8C). This was a good finding, since increased GLP-1 release may be beneficial in the management of obesity. GLP-1 may help control appetite and satiety, which could lead to more controlled food intake and potential weight loss. This finding may explain pA1c®HI mechanism of action.

2.3. pA1c®HI had a Protective Effect Against Liver Injury in HFD-Fed Mice.

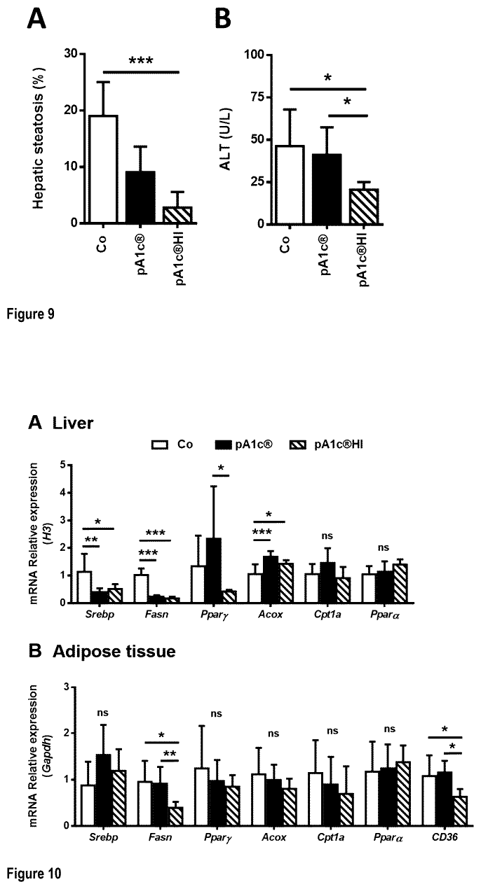

Hepatic steatosis, characterised by the abnormal accumulation of fat in the liver, is a significant contributor to the development of obesity. When we looked at the effect of the probiotic/postbiotic on the liver damage induced by HFD consumption, we found that the administration of the postbiotic generated significant lower levels of steatosis than the Co group (Co: 19.0±6.0% vs. pA1c®HI: 2.8±2.8%; p<0.05), indicating that pA1c®HI was capable of ameliorated hepatic steatosis. Despite the fact that steatosis levels were lower in the pA1c® group vs. Co group, significant differences were not found probably due to data variability (Co: 19.0±6.0% vs. pA1c®: 9.1±4.5%; p>0.05) (FIG. 9A). This finding, once again support the affirmation that pA1c®HI is more effective than pA1c®.

Transaminases level in serum was measured in relation to liver damage. Excessive fat accumulation in the liver, characteristic of obesity, can trigger inflammation and liver damage, which is reflected in increased transaminases in the bloodstream. Remarkably, we found a significant decrease in serum ALT levels in the pA1c®HI group vs. Co group (p<0.05), and pA1c®HI group vs. pA1c® group (p<0.05), having the lowest ALT levels the pA1c®HI group (Co: 46.2±21.8 U/L, pA1c®: 41.1±16.5 U/L, and pA1c®HI: 20.5±4.6 U/L) (FIG. 9B).

2.4. Both pA1c® and pA1c®HI Modulated Hepatic Lipid Metabolism but Only pA1c®HI had an Effect on Adipose Tissue Lipid Metabolism Gene Expression

We analysed the gene expression of several lipid metabolism markers in both in liver and adipose tissue. In terms of liver analysis (FIG. 10A), we can highlight that the expression of de novo lipogenesis (DNL) markers Srebp and Fasn was downregulated in the pA1c® group (p<0.01) and also in the pA1c®HI group (p<0.05) when compared to the Co group, evidencing the implication of both in the de novo lipogenesis process, giving us more information about the surrounded mechanism of action.

Pparγ, a lipid uptake and adipogenic marker, significant differences were found between the pA1c® and pA1c®HI groups (p<0.05). Regarding beta-oxidation pathway markers, although we did not find significant differences for the mitochondrial fatty acid degradation genes Cpt1 or Pparα (p>0.05), we found differences for the peroxisomal fatty acid degradation gene Acox, which expression was higher in both, the pA1c® and pA1c®HI groups compared to the Co group (p<0.001 and p<0.05 respectively) suggesting a beta-oxidation activation in the peroxisome.

In conclusion, both the probiotic and the postbiotic had similar metabolic effects in the liver, decreasing DNL and increasing the catabolic beta-oxidation pathway.

On the other hand, in the adipose tissue (FIG. 10B), DNL marker Fasn decreased only in the pA1c®HI group in comparison with co and pA1c® (pA1c®HI vs. Co, p<0.05; and pA1c®HI vs. pA1c®, p<0.01), and no differences were found for Srebp or the lipid uptake marker Pparγ (p>0.05). Unlike in the liver, no significant differences were found for the beta-oxidation pathway markers (Acox, Cpt1 or Pparα (p>0.05) but we did find significant differences for CD36, being its expression lower in the pA1c®HI group compared to the Co or pA1c® groups (p<0.05). In summary, since CD36 is involved in the import of fatty acids into cells, the administration of the postbiotic decreased the store of fatty acids in adipose tissue cells and DNL.

### CITATION LIST

WO2021123355
Sambrook, J. and Russell, D W “Molecular Cloning: A Laboratory Manual”, Chapter 13, “Mutagenesis”, Cold Spring Harbor, 3rd Ed., 2001
Fan et al. Inactivation of Gram-Positive Bacteria by Novel Phenolic Branched-Chain Fatty Acids. J Food Prot, 2017, vol 80, issue 1, p. 6-14. doi: 10.4315/0362-028X.JFP-16-080.
Levinson et al, Production of potent inactivated vaccines with ultraviolet irradiation: il. an abbreviated preliminary report on sterilization of bacteria and immunization with rabies and St. Louis encephalitis vaccines. JAMA 1944; 125 (8): 531-532. doi: 10.1001/jama.1944.02850260005002.
Hankaniemi et al, Formalin treatment increases the stability and immunogenicity of coxsackievirus B1 VLP vaccine. Antiviral Research 2019, Volume 171, 104595.
EUROPEAN PHARMACOPOEIA 6.0. (2008). 2.9.3. Dissolution test for solid dosage forms. pp. 266-275.
Cabello-Olmo, M.; Oneca, M.; Pajares, M. J.; Jiménez, M.; Ayo, J.; Encío, I. J.; Barajas, M.; Araña, M. Antidiabetic Effects of Pediococcus acidilactici pA1c on HFD-Induced Mice. Nutrients 2022, 14.
Wang, Q.; Garrity, G. M.; Tiedje, J. M.; Cole, J. R. Naïve Bayesian classifier for rapid assignment of rRNA sequences into the new bacterial taxonomy. Appl. Environ. Microbiol. 2007, 73, 5261-5267.
Yavorov-Dayliev D, Milagro F I, Ayo J, Oneca M, Goyache I, López-Yoldi M, Aranaz P. Glucose-lowering effects of a synbiotic combination containing Pediococcus acidilactici in C. elegans and mice. Diabetologia. 2023 November; 66 (11): 2117-2138. doi: 10.1007/s00125-023-05981-w. Epub 2023 Aug. 16.
Yue Y, Li S, Shen P, Park Y. Caenorhabditis elegans as a model for obesity research. Curr Res Food Sci. 2021 Oct. 1; 4:692-697. doi: 10.1016/j.crfs.2021.09.008.
Escorcia W., Ruter D. L., Nhan J., Curran S. P. Quantification of Lipid Abundance and Evaluation of Lipid Distribution in Caenorhabditis elegans by Nile Red and Oil Red O Staining. J. Vis. Exp. 2018; 2018:57352. doi: 10.3791/57352.
Xu A, Zhang Z, Ko S H, Fisher A L, Liu Z, Chen L. Microtubule regulators act in the nervous system to modulate fat metabolism and longevity through DAF-16 in C. elegans. Aging Cell. 2019 April; 18 (2): e12884. doi: 10.1111/acel.12884. Epub 2019 Jan. 14.
Buranasin P, Mizutani K, Iwasaki K, Pawaputanon Na Mahasarakham C, Kido D, et al. (2018) High glucose-induced oxidative stress impairs proliferation and migration of human gingival fibroblasts. PLOS ONE 13 (8): e0201855. https://doi.org/10.1371/journal.pone.0201855

1. A method for treating or preventing obesity or overweight, reducing body fat, preventing fat accumulation, reducing appetite, reducing food intake, or preventing weight gain in an animal in need thereof, including a human, the method comprising administering to said animal in need thereof an effective amount of a postbiotic preparation of Pediococcus acidilactici (P. acidilactici).

2. The method according to claim 1, which is for treating obesity or overweight.

3. The method according to claim 1, wherein the postbiotic preparation comprises inanimate P. acidilactici cells or a lysate of P. acidilactici.

4. The method according to claim 1, wherein the P. acidilactici is selected from the group consisting of P. acidilactici deposited at the “Colección Española de Cultivos Tipo” (CECT) with identification reference CECT 9879, P. acidilactici deposited at the “Colección Española de Cultivos Tipo” (CECT) with identification reference CECT 9889 P. acidilactici deposited at the “Colección Española de Cultivos Tipo” (CECT) with identification reference CECT 9967, and combinations thereof.

5. The method according to claim 4, wherein the P. acidilactici is P. acidilactici CECT 9879.

6. The method according to claim 2, wherein the postbiotic preparation comprises inanimate P. acidilactici cells or a lysate of P. acidilactici CECT 9879.

7. The method according to claim 1, wherein the effective amount of postbiotic preparation is one providing from 10⁵ to 10¹¹ cells of P. acidilactici per day per animal, in particular, from 10⁹ to 10¹¹ cells of P. acidilactici per day per animal, preferably around 10¹⁰ cells of P. acidilactici per day per animal.

8. The method according to claim 1, said method comprising administering to said animal in need thereof a composition comprising an effective amount of a postbiotic preparation of Pediococcus acidilactici.

9. The method according to claim 8, which is for treating obesity or overweight.

10. The method according to claim 8, wherein the composition is an oral composition.

11. The method according to claim 10, wherein the oral composition is a food or feed product, a food or feed ingredient, or a food or feed supplement, and optionally further comprises food or feed additives selected from the group consisting of preservatives, anti-oxidants, emulsifiers, colorants, and bulking agents.

12. The method according to claim 10, wherein the oral composition is a pharmaceutical or nutraceutical composition and also comprises pharmaceutically acceptable excipients and carriers, in particular further comprising a lubricating agent, and/or a bulking agent, and/or an enteric coating.

13. The method according to claim 10, wherein the oral composition further comprises zinc or chromium, in particular, wherein the chromium is chromium picolinate.

14. The method according to claim 9, wherein the composition is an oral composition comprising inanimate P. acidilactici cells or a lysate of P. acidilactici CECT 9879, together with zinc or chromium, in particular, wherein the chromium is chromium picolinate.

15. The method according to claim 1, said method further comprising administering to said animal in need thereof:
(a) an effective amount of a further active ingredient that is effective in preventing and/or treating obesity or overweight, in reducing body fat, in preventing fat accumulation, in reducing appetite, in reducing food intake, in preventing adipogenesis in the adipose tissue, or in preventing weight gain, in particular wherein the further active ingredient is selected from the group consisting of zinc, chromium, Semaglutide, Liraglutide, GLP-1/GIP dual agonists such as tirzepatide, gastrointestinal lipase inhibitor such as Orlistat, appetite suppressants such as phentermine, topiramate, or natural appetite suppressants, Naltrexone/Bupropion, Setmelanotide, Tirzepatide, and combinations thereof; and/or
(b) a low-calorie diet and/or regular exercise.

16. The method according to claim 15, wherein the further active ingredient is of zinc or chromium, in particular, wherein the chromium is chromium picolinate.

17. The method according to claim 16, wherein the effective amount of postbiotic preparation is one providing from 10⁵ to 10¹¹ cells of P. acidilactici per day per animal, the effective amount of Chromium is from 0.5 to 20 μg per day, and the effective amount of Zinc is from 0.1 to 10 mg per day.

18. The method according to claim 17, wherein the postbiotic preparation comprises inanimate P. acidilactici cells or a lysate of P. acidilactici CECT 9879.

19. The method according to claim 17, which is for treating obesity or overweight.

20. The method according to claim 15, wherein the postbiotic preparation and the further active ingredient are administered concomitantly or separately, in any order, within a therapeutically effective interval.
